(19) United States
(12) Patent Application Publication
(10) Pub. No.: US 2017/0368231 A1
(43) Pub. Date: Dec. 28, 2017

(54) Bioengineered Regenerative Graft Matrix, and Methods for Making Thereof

(71) Applicant: DermaGenesis, LLC, Miami, FL (US)

(72) Inventors: Jess Bills, Miami, FL (US); Derek Cox, Miami, FL (US); David Tumey, Miami, FL (US)

(21) Appl. No.: 15/631,628

(22) Filed: Jun. 23, 2017

Related U.S. Application Data
(60) Provisional application No. 62/353,626, filed on Jun. 23, 2016.
(60) Provisional application No. 62/353,620, filed on Jun. 23, 2016.

(51) Int. Cl. A61L 27/60; A61L 27/20; A61L 27/36; A61L 27/56; A61L 27/54; A61L 27/26

(52) U.S. Cl. CPC A61L 27/60; A61L 27/54; A61L 27/26; A61L 27/3633; A61L 27/3687; A61L 27/56; A61L 27/20; A61L 27/3691; A61L 2300/412; A61L 2300/64; A61L 2300/802; A61L 2430/34

(57) ABSTRACT

A skin-substitute is constructed by homogenizing an acellular dermal tissue matrix into a slurry, pouring the slurry into a mold, and lyophilizing the slurry.

### FIELD OF THE INVENTION

The invention relates to technology in support of tissue grafting, and more particularly, skin grafting.

### BACKGROUND OF THE INVENTION

Grafting of skin wounds is frequently necessary to promote healing of large areas of damaged or destroyed skin, such as occurs with serious burns, pressure wounds (bedsores) or intractable wounds such as diabetic ulcers of the skin. A patient's own skin (autograft) can be harvested from an unaffected area and transferred to the wound site. Autografting has the disadvantage of creating a secondary wound, which can be severely detrimental to a patient, thus the availability of skin areas that can be used for autografts is limited. Donor skin (allograft, xenograft or amnion) has been widely used for many years, but immunological compatibility and biosafety issues limit the use of donor skin tissues. Skin allograft tissues can be obtained from cadaver donors by the use of a surgical dermatome, which peels off a very thin (0.015 inch) uniform layer of skin that may be used fresh (after quality assurance testing is completed) or may be placed in cryoprotectant and frozen for several years before grafting. However, allograft or xenograft skin (and amnion tissue) is at best a temporary graft that will be rejected by the recipient patient, usually within 7-21 days, and is associated with permanent scarring that add to the debilitating outcome of extensive wounds.

Other types of temporary skin substitutes are known and used. For example Biobrane® (Dow-Hickham; Sugarland, Tex.) comprises an inner layer of nylon mesh that allows fibro-vascular ingrowth and an outer layer of silastic that provides a vapor and bacterial barrier. However, like donor skin allografts, xenografts and amnion tissue, Biobrane® is a temporary measure and has been associated with permanent scarring of some types of wounds.

The current trend of wound grafting has shifted to a search for substitutes to overcome the deficiencies and limitations of skin auto- and allografts. Bioengineered skin substitutes can be constructed on demand and modulated for specific purposes. In order to promote regeneration of skin, the skin substitute needs to be stable for a sufficient time period (usually about 3 weeks) with a 3D structure that allows ingrowth of blood vessels and fibroblasts, as well as coverage by epithelial cells. After the initial regeneration period, the skin substitute needs to be degraded and removed by host cells without triggering the immune system. To prevent an inflammatory response of the immune system, the materials of a skin substitute must be immunocompatible with the host. Such a skin substitute would thus be considered “permanent”, since it would be incorporated into the healing wound.

In recent years, tissue banks have begun to process sections of donor skin to remove all cellular components from the extracellular matrix (ECM) of the skin. The ECM sections are then purified and lyophilized for long-term storage (up to 5 years) in small fragments, typically about 4 cm×8 cm (32 cm²) and 0.4-0.8 mm thick. The resulting allograft thus comprises an isolated, purified cell-free ECM product derived from dermis that may be rehydrated and applied to a prepared wound bed where it provides a scaffold for migratory cells in the wound margins to infiltrate the allograft and regenerate vascular and dermal tissue. ECM allografts are commercially available to cover areas ranging from about 128-256 cm². For example, see U.S. Pat. No. 8,067,149 B2 issued to Livesey et al., and the product AlloDerm® Tissue Matrix (LifeCell Corporation; Bridgewater N.J.). An alternative form is AlloDerm® Ready to Use, which is non-lyophilized.

Various composites of bovine collagen and/or other ECM components have also been used to produce graft matrices or scaffolds to support human dermal cells in culture to produce skin substitutes. Integra® (Integra LifeSciences Corp.; Plainsboro, N.J.) is a dermal regeneration template comprising bovine collagen, chondroitin-6-sulphate (C6S) and a silastic membrane. Apligraft® (Organogenesis, Inc.; Canton, Mass.) comprises type I bovine collagen, and Matriderm® (Skin and Health Care AG; Billerbeck, Germany) is a synthetic graft matrix of bovine type I collagen with elastin. OrCel® (Fortificell Bioscience, N.Y.) is a bilayered type I bovine collagen sponge. Hyalomatrix® (Fidia Advanced Biopolymers; Padua, Italy) is a bilayer hyaluronan base scaffold. Each of these graft matrices is populated with at least one allogeneic or autologous cell type, such as fibroblast or keratinocyte, to form a skin substitute. However, there are patients who are sensitive to bovine products who would not be able to tolerate bovine-based grafts. Furthermore, human cells have a greater affinity for human ECM components. Thus, the need remains for a graft matrix comprising ECM that is biocompatible with human recipients and provides a hospitable environment for human cells to allow formation of a bioengineered skin substitute for skin grafting and wound healing.

### SUMMARY OF THE INVENTION

The invention in a preferred embodiment provides a method of making a graft matrix useable in a wound, comprising the steps of homogenizing an acellular dermal tissue matrix into a slurry, and pouring the slurry into a mold (such as, e.g., a tray, a petri dish, a culture vessel, a graft matrix carrier; etc.), and lyophilizing the slurry whereby aqueous components are removed, such as, e.g., inventive methods wherein the graft matrix has a surface area at least 10 times greater than the acellular dermal tissue matrix; inventive methods wherein the graft matrix carrier is custom made, comprising a wound-shaped cavity having a size and shape duplicative of an intended recipient's wound, wherein said graft matrix carrier may optionally comprise fenestrations; inventive methods further comprising the steps of: adding a cross-linking agent to the slurry during the homogenizing step, and performing a cross-linking step during the lyophilizing step; inventive methods further comprising performing a cross-linking step after the lyophilizing step; inventive methods wherein the acellular dermal tissue matrix is homogenized in an aqueous solution further comprising at least one acid selected from the group consisting of acetic, sulfuric, nitric, phosphoric, hydrochloric, carbonic, formic, hydrofluoric, perchloric and ascorbic; inventive methods wherein the acellular dermal tissue matrix is homogenized in an aqueous solution further comprising acetic acid wherein the acetic acid is in the range of 1 millimolar to 10 molar; inventive methods comprising use in the homogenizing step of an aqueous solution that comprises at least one salt (such as, e.g., sodium chloride, magnesium chloride, potassium chloride, sodium citrate, sodium acetate, potassium hydroxide, sodium hydroxide, calcium acetate, sodium bicarbonate; etc.); inventive methods comprising use in the homogenizing step of an aqueous solution that comprises at least one salt wherein the salt concentration is in the range of 10 millimolar to 10 molar; inventive methods that further comprise placing the graft matrix in a suitable culture vessel under conditions that permit cells (such as, e.g., fibroblasts, dermal fibroblasts, neonatal foreskin fibroblasts, keratinocytes, dendritic cells, stem cells, adult stem cells, embryonic stem cells, myeloid cells myeloid stem cells, cord blood cells, mesenchymal stem cells, adipose stem cells, neural stem cells, etc.) to infiltrate and populate pores in the graft matrix; inventive methods wherein the acellular dermal tissue matrix is produced from a mammal (such as, e.g., a human); inventive methods further comprising the step of rehydrating the acellular dermal tissue matrix before the homogenizing step; inventive methods wherein in the lyophilizing step, a tray is used wherein the tray is selected from the group consisting of a stainless steel tray, a stainless steel tray comprising an anodized coating, an aluminum tray, an aluminum tray comprising an anodized coating, and a tray comprising an anodized coating; inventive methods wherein in the lyophilizing step, a tray comprising a chromate conversion coating is used; and other inventive methods.

### BRIEF DESCRIPTION OF THE DRAWINGS

FIG. 1 is a flow chart of method steps in an embodiment of the invention.

### DETAILED DESCRIPTION OF THE INVENTION

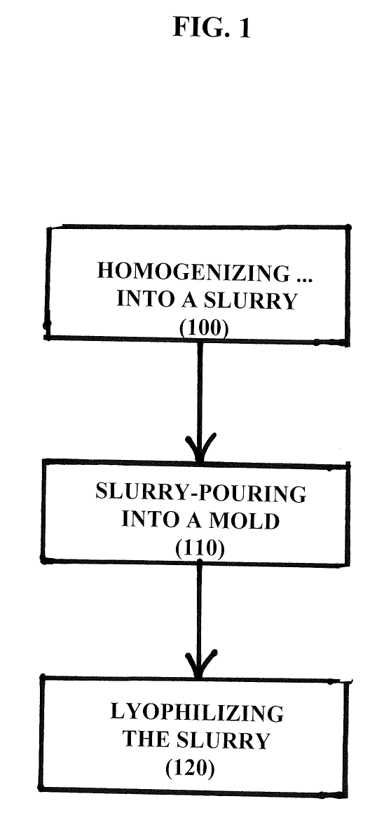

The invention provides a graft matrix suitable for a skin substitute and methods for forming inventive graft matrixes, such as methods according to FIG. 1 comprising steps of homogenizing 100 materials to form a slurry, pouring 110 the slurry into a mold, and lyophilizing 120 the slurry. A skin substitute can be used to cover a wound on a patient in need of a skin graft. The process of wound healing is a complex interplay between the ECM and migrating cells that move from the wound margins into the area in need of tissue repair. In normal wound healing, a clot is formed in a wound, and a superficial clot filling a skin wound is commonly referred to as a scab. The scab covers the wound, sealing out air, reducing fluid loss and providing a barrier to microorganisms. When a wound is too large to form such a barrier, a tissue graft is helpful—sometimes critically so—for healing. In some disorders, such as diabetic ulceration, wounds are refractory to the healing process without grafting.

The bioengineered graft matrix of the invention provides an environment that is conducive to cell migration and tissue regeneration. An inventive graft matrix further provides a scaffold for regeneration and reconstitution of dermal cells to mimic the natural structures of skin. For the purpose of forming a skin substitute, the graft matrix can be seeded with dermal cell types of interest, such as fibroblasts, keratinocytes, dendritic cells, or a variety of stem cell types that can be induced to differentiate into dermal cell types. These cell types can be cultured under conditions that allow migration into the pores of the graft matrix where they can proliferate and populate the graft matrix. The cells may reach confluence within the graft matrix, but are not required to do so to provide a useful skin substitute. A skin substitute prepared according to the methods of the invention can be used to graft a wound and provide a temporary protective barrier over the wound. The presence of the graft edges induce migration of host cells from the wound margins into the graft, and donor cells are replaced as the host cells travel into the graft matrix. The graft matrix materials are biocompatible with the host, and thus are a template for remodeling performed by the host cells during the process of healing and tissue regeneration.

An allograft sometimes is more broadly defined as a donor tissue graft, but in this application “allograft” is used interchangeably with “acellular dermal tissue matrix” and “acellular dermal matrix”.

The preferred starting material for making an inventive graft matrix is an acellular dermal tissue matrix. Allograft products are generally available from a tissue bank, such as AlloSource, CellRight Technologies, Regenerative Biologics, and the University of Miami Tissue Bank, or from other commercial sources. In the case of acellular dermal tissue matrix, an allograft is prepared from dermal tissue using FDA-approved protocols for obtaining, decellularizing and purifying harvested cadaver dermis. The lyophilized or desiccated dermal matrix is cut into uniform pieces and aseptically sealed for long-term storage of up to five years.

Acellular dermal tissue matrices are used in the art of grafting and tissue repair. An allograft is rehydrated in a suitable medium and applied directly to the area to be grafted or repaired. The allograft may be cut or trimmed to fit a wound area, but is otherwise ready to use without further modification. For example, see Wei et al., J Periodontol. 2000 August; 71(8):1297-305, wherein an acellular dermal matrix is used as a periodontal allograft. See also El-Kassaby et al., Ann Maxillofac Surg. 2014 July-December; 4(2):158-61 for discussion of using an allograft for closure of oronasal fistula repair in patients with cleft palate. The use of allografts for grafting and tissue repair does not require transformation of the acellular dermal tissue; rather it promotes healing by covering a wound and allowing host repair processes to work.

The invention provides a method for homogenizing material (such as, e.g., a rehydrated allograft or acellular dermal tissue matrix) and reconstituting the material into a sponge-like material that can be populated with autologous or allogeneic cells of interest prior to grafting. The term “graft matrix” is used herein to refer to a composition that is constructed from homogenized acellular dermal tissue matrix. The same product could be made directly from donor skin, however, access to cadaver donor skin is strictly regulated by the FDA. In practice, one could decellularize donor skin, purify it, and move directly to the homogenization step of the examples provided herein, thus skipping the step of desiccating or lyophilizing the acellular dermal matrix. The practical solution to lack of access to donor skin is to use the allograft product provided by tissue banks. However, it is contemplated that the invention could be made directly from donor skin, such as cadaver skin, using decellularization and purification steps that are well-known in the art.

Human acellular dermal tissue matrix is the preferred starting material for practicing the methods of the invention. However, it should be understood that an acellular dermal tissue matrix derived from a non-human species could also be used in practicing the invention. The sources most likely to be compatible are mammalian, and could include by are not limited to bovine, porcine and primate species. Furthermore, it should be understood that an acellular tissue matrix could be made from other non-dermal tissue types, and these could also be used as a starting material for practicing the invention.

One advantage of reconstituting the allograft into a sponge-like material is that the pores of the graft matrix allow influx of migrating cells that repair damaged tissues during wound healing. The three-dimensional structure of the graft matrix mimics the extra-cellular matrix (ECM) of natural healthy skin. One of the problems of wound healing is scar formation, and a characteristic of scar formation is poorly formed ECM that does not allow reconstruction of normal skin layers. While an allograft comprises ECM, it is only a subset of the full thickness, since it is harvested only from the dermal layer of the donor skin. Reconstituting the allograft into a graft matrix of the invention recapitulates a thickness closer to that of normal healthy skin, while creating a porous network also resembling ECM. The porosity is another advantage of the invention over the allograft starting material, which is compressed during processing and may not fully re-expand when rehydrated.

Another advantage of the invention is that the surface area of an allograft is increased at least 10-fold by homogenization and further steps of the method. The method can be likened somewhat to the process of making cotton candy, wherein a small amount of sugar and stabilizing agents are whipped into a state wherein the sugar is transformed into a radically increased volume of sugar-containing fibers. The inventors have found that a 32 cm² piece of allograft can be transformed into a graft matrix of 300 cm² or more, with the thickness also increased by a similar factor.

The homogenized slurry of starting materials can be poured or aliquoted into molds of any shape or size desired, limited only by the quantity of slurry and capacity for lyophilization. A mold may be as simple as wells in a culture vessel, petri dishes, a tray with raised edges, or a mold made specifically for the purpose of forming a graft matrix. In one embodiment, a graft matrix is made in a uniform square, rectangular or circular shape and subsequently trimmed or cut to a desired shape for culturing or for grafting. In another embodiment, a graft matrix is formed in a mold or graft carrier that has been custom designed to replicate the size and shape of a wound to be covered, filled or grafted.

The term “extracellular matrix” or “ECM” refers to noncellular molecules that surround cells in a naturally-occurring tissue, such as skin, but are also found in many other tissues of the body. ECM components include chondroitin 6-sulfate, hyaluronic acid, fibronectin, growth factors, integrin binding sites, collagen, mucopolysaccharides, glucosamines, elastin and tenascin. Chondroitin 6-Sulfate (C6S) attaches to collagen and allows cells to more easily adhere or move.

In one embodiment, the graft matrix of the invention comprises C6S, which is homogenized with the acellular dermal tissue matrix. C6S is available commercially, most commonly derived from bovine and marine species, but from others as well. Without being bound to theory, C6S is incorporated in the graft matrix of the invention to promote cell adhesion and migration during culture of autologous or allogenic cells and to promote host cell migration and residence into and within the graft. Thus C6S contributes to the hospitable environment for cell proliferation within the graft matrix of the invention. Useful C6S concentration can be in the range of 0.001-25%.

In other embodiments, addition of other ECM components is contemplated, with the goal of enhancing the stability of the graft matrix and promoting cell migration and expansion. Collagen can also be used as a component of the graft matrix. Collagen's bioactive properties remain bioactive when the triple helical secondary structure is preserved. When the secondary structure is disrupted, the collagen is converted into gelatin. Therefore, it is important to maintain the secondary structure when incorporating collagen into the slurry used to make a graft matrix of the invention.

Hyaluronic acid (HA) is the first molecule to appear during wound healing, signals cells to initiate “wound healing mode,” signals cells to proliferate and migrate to close wound. In some embodiments, HA may be incorporated into the slurry and thus are components of the graft matrix of the invention. Useful HA concentrations can be in the range of 0.001-25%.

Fibronectin is secreted by fibroblasts and plays a major role in cell adhesion, migration and growth. In some embodiments, fibronectin may be incorporated into the slurry and thus are components of the graft matrix of the invention. Useful fibronectin concentrations can be in the range of 0.001-10%.

Tenascin appears in healing wounds and plays a role in cell migration. In some embodiments, tenascin may be incorporated into the slurry and thus are components of the graft matrix of the invention. Useful tenascin concentrations can be in the range of 0.001-10%.

Elastin or elastic fibers are used as mechanical properties of the ECM and allows many tissues in the body to resume their shape after stretching or contracting. In some embodiments, elastin may be incorporated into the slurry and thus are components of the graft matrix of the invention. Useful elastin concentrations can be in the range of 0.001-10%.

The starting material of acellular dermal tissue matrix is rehydrated in a weak solution of acid to prepare it for homogenization prior to mixing it into the slurry of cellular dermal tissue matrix. It would be readily understood by one of ordinary skill in the art that any of many weak acids may be substituted. For example, any of sulfuric, nitric, phosphoric, hydrochloric, carbonic, formic, hydrofluoric, ascorbic and perchloric acid may be used in place of acetic acid in the practice of the invention. Mixtures of any of these acids are also contemplated. In addition, there is a wide range of acid concentrations that may be used. In some applications, acids can shorten the lifespan of lyophilization equipment, so use of a mild acid is preferred for that reason. The range of concentration can be as low as 1 mM to 10 M, depending on the solvent. In any case, a good rule of thumb is that the acid solution must be strong enough to promote dissolution of collagen without disrupting the secondary structure.

In some embodiments, a salt is added prior to or during homogenization. Without being bound to theory, the salt enhances solubility of the components during homogenization. It would be readily understood by one of ordinary skill in the art that any one of many salts may be substituted. For example, any of sodium chloride, magnesium chloride, potassium chloride, sodium citrate, sodium acetate, potassium hydroxide, sodium hydroxide, calcium acetate, and sodium bicarbonate, or any combination of these, may be used in place of sodium chloride in the practice of the invention. In addition, there is a wide range of salt concentrations that may be used.

Crosslinking agents are used to bond the proteins of the graft matrix together, thus creating a stronger matrix scaffold that is resistant to handling. Furthermore, when fibroblasts migrate into the graft matrix of the invention, they will remodel it to their liking, and without crosslinking the graft matrix will fall apart in several days. 1-ethyl-3-(-3-dimethylaminopropyl) carbodiimide (EDC) is a commonly used crosslinker for ECM components, such as collagen, C6S, and HA. Useful concentrations of EDC can be in the range of 1 mM to 1M, 20 mM to 50 mM, 10 mM to 60 mM, and 5 mM to 100 mM. In addition to EDC, there are other agents that act as chemical bonders for the matrix, such as: N-hydroxysuccinimide (NHS); glutaraldehyde; even certain forms of gamma-irradiation and dehydrothermal crosslinking.

Lyophilization is a process wherein a solid (frozen acetic acid and ice in this case) is removed from a solution while avoiding the liquid phase, thus going from solid directly to gas. This is critical since heat would desiccate the graft matrix and collapse the pores. The time for this process varies with the amount of liquid that needs to be removed, and will increase with an increased thickness or quantity of slurry. The actual time to lyophilize to dryness may be less than 12 hours, while some slurry formulations will need to lyophilize for at least 20-30 hours, up to 72 hours.

The following examples are provided to more fully disclose and describe practice of the invention.

### Example 1. Homogenizing a Slurry of Acellular Dermal Tissue Matrix

1) Add approximately 0.5 g acellular dermal tissue matrix to approximately 100 mL of 50 mM acetic acid. The concentration of acellular dermal tissue matrix is about 0.5%. In some experiments, more or less tissue is added, and 0.1-1% collagen is added
2) Allow acellular dermal tissue matrix to rehydrate in acetic acid solution for approximately 5-20 minutes.
3) Homogenize at 24,000 rpm for approximately 2 hours in the cold (e.g., in an ice bath or cold room). Homogenization can be continuous or intermittent (i.e., 30 minutes of homogenization followed by 10 minutes resting, times 4).
4) Mix 50 mg chondroitin 6-sulfate (C6S) to 50 mL of 50 mM acetic acid; concentration of C6S will be 0.1%.
5) Place C6S solution in an IV bag or peristaltic pump apparatus to control the rate of the drip.
6) While homogenizing the acellular dermal tissue matrix mix, begin adding C6S mix dropwise to slurry at a rate of 150 mL/hour until all 50 mL of C6S has been added. Final concentration of new solution is approximately 0.5% acellular dermal tissue matrix and 0.02% C6S. Note: if the C6S solution begins to mix enter the homogenization vessel at the beginning of step 4, all the C6S will be added by about halfway through the homogenization process.

### Example 2. Homogenizing a Slurry with Acellular Dermal Tissue and Additional ECM Components

1) Add approximately 0.5 g acellular dermal tissue matrix to approximately 100 mL of 50 mM acetic acid. The concentration of acellular dermal tissue matrix is about 0.5%. In some experiments, more or less tissue is added, and 0.1-1% collagen is added
2) Allow acellular dermal tissue matrix to rehydrate in acetic acid solution for approximately 5-20 minutes.
3) Homogenize at 24,000 rpm for approximately 2 hours in the cold (e.g., in an ice bath or cold room). Homogenization can be continuous or intermittent (i.e., 30 minutes of homogenization followed by 10 minutes resting, times 4).
4) Mix 50 mg chondroitin 6-sulfate (C6S) to 50 mL of 50 mM acetic acid; concentration of C6S will be 0.1%.
5) Place C6S solution in an IV bag or peristaltic pump apparatus to control the rate of the drip.
6) While homogenizing the acellular dermal tissue matrix mix, begin adding C6S mix dropwise to slurry at a rate of 150 mL/hour until all 50 mL of C6S has been added. Final concentration of new solution is approximately 0.5% acellular dermal tissue matrix and 0.02% C6S. Note: if the C6S solution begins to mix enter the homogenization vessel at the beginning of step 4, all the C6S will be added by about halfway through the homogenization process.
   Additional ECM components are added to the slurry. Examples of additional components to add are, e.g., glycosaminoglycans (GAG) (such as HA or fibronectin), proteins (such as elastin or tenascin) or combinations thereof. In this example, HA and fibronectin are selected for inclusion.
7) Add 100 mg hyaluronic acid (0.04%) to the 250 mL acellular dermal tissue matrix+C6S mix
8) Homogenize for about 10 seconds or until the HA dissolves in the acetic acid.
9) Add 2.5 mg fibronectin for a final concentration of 0.001%.
10) Homogenize for about 10 seconds or until fibronectin dissolves in the acetic acid.
11) Continue to homogenize the final mixture for remainder of homogenization time at 24,000 rpms. A total of 2 hours (see step 4) is usually sufficient.

### Example 3. Forming the Graft Matrix

The slurry formed in Examples 1 or 2 is sufficiently fluid so as to take the shape of any mold or carrier. The depth of the slurry when instilled in the mold or carrier will determine the thickness of the final graft matrix. In some embodiments, a carrier may comprise fenestrations or “teeth” to hold the graft matrix in place during lyophylization.

A 6 cm petri dish will generate a graft matrix with a surface area of about 21 cm², and 10 mL of slurry will dry down to yield a graft matrix that is approximately 2-5 mm thick. Pour 10 mL of slurry into each 6 cm petri dish.
1) Turn on the lyophilizer and set the shelf temperature to −45 degrees Celsius (temperature controls pore size, the colder the initial temperature the smaller the pores. −45 C gives average pore sizes between 70 uM+/−30 uM which is ideal for fibroblasts to migrate through and inhabit). The machine may take several hours to reach a uniform shelf temperature of −45 C.
2) Place the molds (in this example, the petri dishes) onto the shelf and allow to freeze until sample is completely frozen. In some instances, the freezing step has taken at least 2 hours.
3) Turn on the vacuum, and increase the temperature to −2 degrees C. and allow to lyophilize to dryness. In some instances, drying is complete with an overnight lyophilization. When ready to remove samples, increase shelf temperature to 20 C, approx. room temperature.
4) Once temperature has been achieved, turn the vacuum off and the lyophilizer off, remove samples. Note: Do not leave them in a warm area, they are fragile until crosslinked.

### Example 4. Crosslinking Performed after Lyophilization

1) Remove molded and lyophilized graft matrices from lyophilizer, maintaining at room temperature or cooler.
2) For a graft matrix molded in a single 6 cm petri dish, add 10-11 mL of 20 mM EDC/absolute ethanol to a graft matrix formed from 10 mL of lyophilized slurry, ensuring that the graft matrix is completely covered.
3) Allow to sit in the EDC solution for 20 hours or overnight.
   Note: Ethanol will evaporate at warmer temperatures and this can collapse the graft matrix, so continue to maintain the graft matrices at room temperature or colder.
4) Remove EDC, wash for 60 minutes with 50% ethanol/H2O, followed by at least three additional washes of distilled water for 15 minutes each.
   Note: in some cases, approximately 50% of the HA will be lost during the wash steps. If a higher HA content is desired, this can be corrected by starting the process with twice as much HA as intended in final product.
5) Place molded matrices back into the lyophilizer on a tray at −45 C and perform a second lyophilization to remove any residual water.

### Example 4A

Crosslinking useable in the invention is not limited to crosslinking using EDC. For example, dehydrothermal (DHT) treatment as a cross linker has been used, such as, e.g., the matrix is baked in a vacuum oven at 100-150 degrees C. for 12-72 hours.

### Example 5. Crosslinking Performed During First Lyophilization Step

A crosslinking agent is added to the slurry formed in step 6 of Example 1 or 2. The slurry is instilled into molds or carriers for the lyophilization step. The lyophylization step is performed as described in Example 3, with the temperature adjusted to allow sufficient time for crosslinking to occur during lyophilization. The temperature will vary according to the crosslinking agent used, and also because of the absolute ethanol content.

### Example 6 (Vitamins)

In this example, at least one vitamin (such as, e.g., vitamin A, vitamin C, vitamin D, a combination thereof, etc.) is infused into the slurry, and remains in the completed graft matrix useable in a wound after the lyophilization step.

### Example 7 (GAGs)

Examples of GAGs useable in the invention are, e.g., chitosan; heparan sulfate; keratan sulfate; dermatan sulfate; and combinations thereof, alone or in combination with one or more of Hyaluronic acid, Heparin, and Chondroitin sulfate.

### Example 8

In this example, taking into account that allografts can have a range of thickness, 2.5 g of the human allograft was weighed out, and that produced 500 ml of slurry at a collagen % of 0.5%. That 500 ml of slurry would make (roughly) an 11×15 inch matrix (roughly 28×38 cm).

### Example 9 (Using a Metal Tray with Anodized Coating in Lyophilization Step)

In this example, a metal tray comprising an anodized coating (such as a metal tray made to order by a metal-working shop) is used in the lyophilizing step, such as:

9A. An aluminum tray comprising an anodized coating.
9B. An aluminum tray comprising a chromate conversion coating.
9C. A stainless steel tray comprising an anodized coating.
9D. A metal tray comprising an anodized coating.
9E. A tray comprising a chromate conversion coating.
9F. An aluminum tray comprising 606TI chromate conversion coating as the anodized coating was used in the lyophilizing step of a matrix production process. These aluminum trays comprising the 6061TI chromate conversion coating are associated with a spectacular, unexpected result, of influencing capacity to manufacture matrices with more precise pore sizes.

The above described embodiments are set forth by way of example and are not limiting. It will be readily apparent that obvious modification, derivations and variations can be made to the embodiments. The claims appended hereto should be read in their full scope including any such modifications, derivations and variations.

What is claimed is:

1. A method of making a graft matrix useable in a wound, comprising the steps of
a. homogenizing an acellular dermal tissue matrix into a slurry, and
b. pouring the slurry into a mold, and
c. lyophilizing the slurry whereby aqueous components are removed.

2. The method of claim 1, wherein the graft matrix has a surface area at least 10 times greater than the acellular dermal tissue matrix.

3. The method of claim 1, wherein the mold is selected from the group consisting of a tray, a petri dish, a culture vessel and graft matrix carrier.

4. The method of claim 3, wherein the graft matrix carrier is custom made, comprising a wound-shaped cavity having a size and shape duplicative of an intended recipient's wound, wherein said graft matrix carrier may optionally comprise fenestrations.

5. The method of claim 1, further comprising the steps of
a. adding a cross-linking agent to the slurry during the homogenizing step, and
b. performing a cross-linking step during the lyophilizing step.

6. The method of claim 1, further comprising performing a cross-linking step after the lyophilizing step.

7. The method of claim 1, wherein the acellular dermal tissue matrix is homogenized in an aqueous solution further comprising at least one acid selected from the group consisting of acetic, sulfuric, nitric, phosphoric, hydrochloric, carbonic, formic, hydrofluoric, perchloric and ascorbic.

8. The method of claim 7, wherein the acid is acetic.

9. The method of claim 8, wherein the acetic acid is in the range of 1 millimolar to 10 molar.

10. The method of claim 7, wherein the aqueous solution further comprises at least one salt selected from the group consisting of sodium chloride, magnesium chloride, potassium chloride, sodium citrate, sodium acetate, potassium hydroxide, sodium hydroxide, calcium acetate, and sodium bicarbonate.

11. The method of claim 10, wherein the salt concentration is in the range of 10 millimolar to 10 molar.

12. The method of claim 1, further comprising the steps of placing the graft matrix in a suitable culture vessel under conditions that permit cells to infiltrate and populate pores in the graft matrix.

13. The method of claim 12, wherein the cells are selected from the group consisting of fibroblasts, dermal fibroblasts, neonatal foreskin fibroblasts, keratinocytes, dendritic cells, stem cells, adult stem cells, embryonic stem cells, myeloid cells myeloid stem cells, cord blood cells, mesenchymal stem cells, adipose stem cells, and neural stem cells.

14. The method of claim 1, further comprising the step of rehydrating the acellular dermal tissue matrix before the homogenizing step.

15. The method of claim 1, further comprising a step of infusing at least one vitamin into the slurry.

16. The method of claim 1, wherein the slurry comprises at least one GAG.

17. The method of claim 16, wherein the GAG is selected from the group consisting of: Hyaluronan; Hyaluronic acid; Heparin; chondroitin sulfate; chitosan; heparin sulfate; Keratan sulfate; and Dermatan sulfate.

18. The method of claim 1, further comprising a crosslinking step selected from the group consisting of crosslinking in which EDC is used and crosslinking in which dehydrothermal treatment is used.

19. The method of claim 1, wherein in the lyophilizing step, a tray is used wherein the tray is selected from the group consisting of a stainless steel tray, a stainless steel tray comprising an anodized coating, an aluminum tray, an aluminum tray comprising an anodized coating, and a tray comprising an anodized coating.

20. The method of claim 1, wherein in the lyophilizing step, a tray comprising a chromate conversion coating is used.
